SEQUENCE LISTING (123 sequences)

SEQ ID NO: 1 - RNA, 110 nt, Homo sapiens
cuggauacagaguggaccggcuggccccaucuggaagacuagugauuuuguuguugucuuacugcgcucaacaacaaaucccagucuaccuaauggugccagccaucgca

SEQ ID NO: 2 - RNA, 23 nt, Homo sapiens
uggaagacuagugauuuuguugu

SEQ ID NO: 3 - RNA, 22 nt, Homo sapiens
caacaaaucccagucuaccuaa

SEQ ID NO: 4 - RNA, 98 nt, Homo sapiens
uugaggccuuaaaguacuguagcagcacaucaugguuuacaugcuacagucaagaugcgaaucauuauuugcugcucuagaaauuuaaggaaauucau

SEQ ID NO: 5 - RNA, 22 nt, Homo sapiens
uagcagcacaucaugguuuaca

SEQ ID NO: 6 - RNA, 22 nt, Homo sapiens
cgaaucauuauuugcugcucua

SEQ ID NO: 7 - RNA, 89 nt, Homo sapiens
gucagcagugccuuagcagcacguaaauauuggcguuaagauucuaaaauuaucuccaguauuaacugugcugcugaaguaagguugac

SEQ ID NO: 8 - RNA, 81 nt, Homo sapiens
guuccacucuagcagcacguaaauauuggcguagugaaauauauauuaaacaccaauauuacugugcugcuuuagugugac

SEQ ID NO: 9 - RNA, 22 nt, Homo sapiens
uagcagcacguaaauauuggcg

SEQ ID NO: 10 - RNA, 22 nt, Homo sapiens
ccaauauuacugugcugcuuua

SEQ ID NO: 11 - RNA, 84 nt, Homo sapiens
gucagaauaaugucaaagugcuuacagugcagguagugauaugugcaucuacugcagugaaggcacuuguagcauuauggugac

SEQ ID NO: 12 - RNA, 23 nt, Homo sapiens
caaagugcuuacagugcagguag

SEQ ID NO: 13 - RNA, 22 nt, Homo sapiens
acugcagugaaggcacuuguag

SEQ ID NO: 14 - RNA, 87 nt, Homo sapiens
cacuguucuaugguuaguuuugcagguuugcauccagcugugugauauucugcugugcaaauccaugcaaaacugacugugguagug

SEQ ID NO: 15 - RNA, 23 nt, Homo sapiens
aguuuugcagguuugcauccagc

SEQ ID NO: 16 - RNA, 96 nt, Homo sapiens
acauugcuacuuacaauuaguuuugcagguuugcauuucagcguauauauguauauguggcugugcaaauccaugcaaaacugauugugauaaugu

SEQ ID NO: 17 - RNA, 22 nt, Homo sapiens
aguuuugcagguuugcauuuca

SEQ ID NO: 18 - RNA, 23 nt, Homo sapiens
ugugcaaauccaugcaaaacuga

SEQ ID NO: 19 - RNA, 72 nt, Homo sapiens
ugucggguagcuuaucagacugauguugacuguugaaucucauggcaacaccagucgaugggcugucugaca

SEQ ID NO: 20 - RNA, 22 nt, Homo sapiens
uagcuuaucagacugauguuga

SEQ ID NO: 21 - RNA, 21 nt, Homo sapiens
caacaccagucgaugggcugu

SEQ ID NO: 22 - RNA, 86 nt, Homo sapiens
gguccuugcccucaaggagcucacagucuauugaguuaccuuucugacuuucccacuagauugugagcuccuggagggcaggcacu

SEQ ID NO: 23 - RNA, 22 nt, Homo sapiens
aaggagcucacagucuauugag

SEQ ID NO: 24 - RNA, 22 nt, Homo sapiens
cacuagauugugagcuccugga

SEQ ID NO: 25 - RNA, 71 nt, Homo sapiens
gcgacuguaaacauccucgacuggaagcugugaagccacagaugggcuuucagucggauguuugcagcugc

SEQ ID NO: 26 - RNA, 22 nt, Homo sapiens
uguaaacauccucgacuggaag

SEQ ID NO: 27 - RNA, 22 nt, Homo sapiens
cuuucagucggauguuugcagc

SEQ ID NO: 28 - RNA, 88 nt, Homo sapiens
accaaguuucaguucauguaaacauccuacacucagcuguaauacauggauuggcugggagguggauguuuacuucagcugacuugga

SEQ ID NO: 29 - RNA, 22 nt, Homo sapiens
uguaaacauccuacacucagcu

SEQ ID NO: 30 - RNA, 22 nt, Homo sapiens
cugggagguggauguuuacuuc

SEQ ID NO: 31 - RNA, 89 nt, Homo sapiens
accaugcuguaguguguguaaacauccuacacucucagcugugagcucaagguggcugggagaggguuguuuacuccuucugccaugga

SEQ ID NO: 32 - RNA, 22 nt, Homo sapiens
cugggagaggguuguuuacucc

SEQ ID NO: 33 - RNA, 72 nt, Homo sapiens
agauacuguaaacauccuacacucucagcuguggaaaguaagaaagcugggagaaggcuguuuacucuuucu

SEQ ID NO: 34 - RNA, 23 nt, Homo sapiens
uguaaacauccuacacucucagc

SEQ ID NO: 35 - RNA, 22 nt, Homo sapiens
cugggagaaggcuguuuacucu

SEQ ID NO: 36 - RNA, 70 nt, Homo sapiens
guuguuguaaacauccccgacuggaagcuguaagacacagcuaagcuuucagucagauguuugcugcuac

SEQ ID NO: 37 - RNA, 22 nt, Homo sapiens
uguaaacauccccgacuggaag

SEQ ID NO: 38 - RNA, 22 nt, Homo sapiens
cuuucagucagauguuugcugc

SEQ ID NO: 39 - RNA, 84 nt, Homo sapiens
gugcucgguuuguaggcagugucauuagcugauuguacuguggugguuacaaucacuaacuccacugccaucaaaacaaggcac

SEQ ID NO: 40 - RNA, 23 nt, Homo sapiens
uaggcagugucauuagcugauug

SEQ ID NO: 41 - RNA, 22 nt, Homo sapiens
caaucacuaacuccacugccau

SEQ ID NO: 42 - RNA, 78 nt, Homo sapiens
cuuucuacacagguugggaucgguugcaaugcuguguuucuguaugguauugcacuugucccggccuguugaguuugg

SEQ ID NO: 43 - RNA, 23 nt, Homo sapiens
agguugggaucgguugcaaugcu

SEQ ID NO: 44 - RNA, 22 nt, Homo sapiens
uauugcacuugucccggccugu

SEQ ID NO: 45 - RNA, 75 nt, Homo sapiens
ucaucccuggguggggauuuguugcauuacuuguguucuauauaaaguauugcacuugucccggccuguggaaga

SEQ ID NO: 46 - RNA, 22 nt, Homo sapiens
ggguggggauuuguugcauuac

SEQ ID NO: 47 - RNA, 75 nt, Homo sapiens
ugcccuggcucaguuaucacagugcugaugcugucuauucuaaagguacaguacugugauaacugaaggauggca

SEQ ID NO: 48 - RNA, 22 nt, Homo sapiens
caguuaucacagugcugaugcu

SEQ ID NO: 49 - RNA, 21 nt, Homo sapiens
uacaguacugugauaacugaa

SEQ ID NO: 50 - RNA, 79 nt, Homo sapiens
acuguccuuuuucgguuaucaugguaccgaugcuguauaucugaaagguacaguacugugauaacugaagaaugguggu

SEQ ID NO: 51 - RNA, 21 nt, Homo sapiens
uacaguacugugauaacugaa

SEQ ID NO: 52 - RNA, 81 nt, Homo sapiens
ccuuggccauguaaaagugcuuacagugcagguagcuuuuugagaucuacugcaauguaagcacuucuuacauuaccaugg

SEQ ID NO: 53 - RNA, 23 nt, Homo sapiens
aaaagugcuuacagugcagguag

SEQ ID NO: 54 - RNA, 22 nt, Homo sapiens
cugcaauguaagcacuucuuac

SEQ ID NO: 55 - RNA, 85 nt, Homo sapiens
cgcuggcgacgggacauuauuacuuuugguacgcgcugugacacuucaaacucguaccgugaguaauaaugcgccguccacggca

SEQ ID NO: 56 - RNA, 21 nt, Homo sapiens
cauuauuacuuuugguacgcg

SEQ ID NO: 57 - RNA, 22 nt, Homo sapiens
ucguaccgugaguaauaaugcg

SEQ ID NO: 58 - RNA, 88 nt, Homo sapiens
acaaugcuuugcuagagcugguaaaauggaaccaaaucgccucuucaauggauuugguccccuucaaccagcuguagcuaugcauuga

SEQ ID NO: 59 - RNA, 102 nt, Homo sapiens
gggagccaaaugcuuugcuagagcugguaaaauggaaccaaaucgacuguccaauggauuugguccccuucaaccagcuguagcugugcauugauggcgccg

SEQ ID NO: 60 - RNA, 22 nt, Homo sapiens
uuugguccccuucaaccagcug

SEQ ID NO: 61 - RNA, 100 nt, Homo sapiens
ugugucucucucuguguccugccagugguuuuacccuaugguagguuacgucaugcuguucuaccacaggguagaaccacggacaggauaccggggcacc

SEQ ID NO: 62 - RNA, 22 nt, Homo sapiens
cagugguuuuacccuaugguag

SEQ ID NO: 63 - RNA, 21 nt, Homo sapiens
uaccacaggguagaaccacgg

SEQ ID NO: 64 - RNA, 87 nt, Homo sapiens
gacagugcagucacccauaaaguagaaagcacuacuaacagcacuggaggguguaguguuuccuacuuuauggaugaguguacugug

SEQ ID NO: 65 - RNA, 21 nt, Homo sapiens
cauaaaguagaaagcacuacu

SEQ ID NO: 66 - RNA, 23 nt, Homo sapiens
uguaguguuuccuacuuuaugga

SEQ ID NO: 67 - RNA, 86 nt, Homo sapiens
uggggcccuggcugggauaucaucauauacuguaaguuugcgaugagacacuacaguauagaugauguacuaguccgggcaccccc

SEQ ID NO: 68 - RNA, 22 nt, Homo sapiens
ggauaucaucauauacuguaag

SEQ ID NO: 69 - RNA, 20 nt, Homo sapiens
uacaguauagaugauguacu

SEQ ID NO: 70 - RNA, 88 nt, Homo sapiens
caccuuguccucacgguccaguuuucccaggaaucccuuagaugcuaagauggggauuccuggaaauacuguucuugaggucaugguu

SEQ ID NO: 71 - RNA, 23 nt, Homo sapiens
guccaguuuucccaggaaucccu

SEQ ID NO: 72 - RNA, 22 nt, Homo sapiens
ggauuccuggaaauacuguucu

SEQ ID NO: 73 - RNA, 68 nt, Homo sapiens
gaggcaaaguucugagacacuccgacucugaguaugauagaagucagugcacuacagaacuuugucuc

SEQ ID NO: 74 - RNA, 22 nt, Homo sapiens
aaaguucugagacacuccgacu

SEQ ID NO: 75 - RNA, 22 nt, Homo sapiens
ucagugcacuacagaacuuugu

SEQ ID NO: 76 - RNA, 75 nt, Homo sapiens
ggcugugccggguagagagggcagugggagguaagagcucuucacccuucaccaccuucuccacccagcauggcc

SEQ ID NO: 77 - RNA, 23 nt, Homo sapiens
cggguagagagggcagugggagg

SEQ ID NO: 78 - RNA, 22 nt, Homo sapiens
uucaccaccuucuccacccagc

SEQ ID NO: 79 - RNA, 95 nt, Homo sapiens
ccagcucgggcagccguggccaucuuacugggcagcauuggauggagucaggucucuaauacugccugguaaugaugacggcggagcccugcacg

SEQ ID NO: 80 - RNA, 22 nt, Homo sapiens
caucuuacugggcagcauugga

SEQ ID NO: 81 - RNA, 22 nt, Homo sapiens
uaauacugccugguaaugauga

SEQ ID NO: 82 - RNA, 110 nt, Homo sapiens
aaagauccucagacaauccaugugcuucucuuguccuucauuccaccggagucugucucauacccaaccagauuucaguggagugaaguucaggaggcauggagcugaca

SEQ ID NO: 83 - RNA, 22 nt, Homo sapiens
uccuucauuccaccggagucug

SEQ ID NO: 84 - RNA, 21 nt, Homo sapiens
gauuucaguggagugaaguuc

SEQ ID NO: 85 - RNA, 110 nt, Homo sapiens
acccggcagugccuccaggcgcagggcagccccugcccaccgcacacugcgcugccccagacccacugugcgugugacagcggcugaucugugccugggcagcgcgaccc

SEQ ID NO: 86 - RNA, 22 nt, Homo sapiens
cugugcgugugacagcggcuga

SEQ ID NO: 87 - RNA, 110 nt, Homo sapiens
ccgccccgggccgcggcuccugauuguccaaacgcaauucucgagucuauggcuccggccgagaguugagucuggacgucccgagccgccgcccccaaaccucgagcggg

SEQ ID NO: 88 - RNA, 21 nt, Homo sapiens
ugauuguccaaacgcaauucu

SEQ ID NO: 89 - RNA, 22 nt, Homo sapiens
agaguugagucuggacgucccg

SEQ ID NO: 90 - RNA, 110 nt, Homo sapiens
ugaacauccaggucuggggcaugaaccuggcauacaauguagauuucuguguucguuaggcaacagcuacauugucugcuggguuucaggcuaccuggaaacauguucuc

SEQ ID NO: 91 - RNA, 22 nt, Homo sapiens
accuggcauacaauguagauuu

SEQ ID NO: 92 - RNA, 23 nt, Homo sapiens
agcuacauugucugcuggguuuc

SEQ ID NO: 93 - RNA, 82 nt, Homo sapiens
gcuucgcuccccuccgccuucucuucccgguucuucccggagucgggaaaagcuggguugagagggcgaaaaaggaugaggu

SEQ ID NO: 94 - RNA, 22 nt, Homo sapiens
aaaagcuggguugagagggcga

SEQ ID NO: 95 - RNA, 79 nt, Homo sapiens
aauuaaucccucucuuucuaguucuuccuagagugaggaaaagcuggguugagagggcaaacaaauuaacuaauuaauu

SEQ ID NO: 96 - RNA, 138 nt, Homo sapiens
uguuauuuuuugucuucuaccuaagaauucugucucuuaggcuuucucuucccagauuucccaaaguugggaaaagcuggguugagagggcaaaaggaaaaaaaaagaauucugucucugacauaauuagauagggaa

SEQ ID NO: 97 - RNA, 22 nt, Homo sapiens
aaaagcuggguugagagggcaa

SEQ ID NO: 98 - RNA, 88 nt, Homo sapiens
uuugcauuaaaaaugaggccuucucuucccaguucuucccagagucaggaaaagcuggguugagaggguagaaaaaaaaugauguagg

SEQ ID NO: 99 - RNA, 50 nt, Homo sapiens
cuucucuuuccaguucuucccagaauugggaaaagcuggguugagagggu

SEQ ID NO: 100 - RNA, 20 nt, Homo sapiens
aaaagcuggguugagagggu

SEQ ID NO: 101 - RNA, 48 nt, Homo sapiens
uucucgucccaguucuucccaaaguugagaaaagcuggguugagagga

SEQ ID NO: 102 - RNA, 48 nt, Homo sapiens
uucucuucccaguucuucuuggagucaggaaaagcuggguugagagga

SEQ ID NO: 103 - RNA, 19 nt, Homo sapiens
aaaagcuggguugagagga

SEQ ID NO: 104 - RNA, 53 nt, Homo sapiens
gccuucucuucccaguucuuccuggagucggggaaaagcuggguugagaaggu

SEQ ID NO: 105 - RNA, 18 nt, Homo sapiens
aaagcuggguugagaagg

SEQ ID NO: 106 - RNA, 83 nt, Homo sapiens
cugacuaugccuccccgcauccccuagggcauugguguaaagcuggagacccacugccccaggugcugcuggggguuguaguc

SEQ ID NO: 107 - RNA, 23 nt, Homo sapiens
cgcauccccuagggcauuggugu

SEQ ID NO: 108 - RNA, 20 nt, Homo sapiens
acugccccaggugcugcugg

SEQ ID NO: 109 - RNA, 83 nt, Homo sapiens
cgccggccgaugggcgucuuaccagacaugguuagaccuggcccucugucuaauacugucugguaaaaccguccauccgcugc

SEQ ID NO: 110 - RNA, 22 nt, Homo sapiens
uaauacugucugguaaaaccgu

SEQ ID NO: 111 - RNA, 72 nt, Homo sapiens
cuugggaauggcaaggaaaccguuaccauuacugaguuuaguaaugguaaugguucucuugcuauacccaga

SEQ ID NO: 112 - RNA, 22 nt, Homo sapiens
aaaccguuaccauuacugaguu

SEQ ID NO: 113 - RNA, 68 nt, Homo sapiens
uggguauagcaagagaaccauuaccauuacuaaacucaguaaugguaacgguuuccuugccauuccca

SEQ ID NO: 114 - RNA, 22 nt, Homo sapiens
uagcaagagaaccauuaccauu

SEQ ID NO: 115 - RNA, 68 nt, Homo sapiens
gcauccuguacugagcugccccgaggcccuucaugcugcccagcucggggcagcucaguacaggauac

SEQ ID NO: 116 - RNA, 22 nt, Homo sapiens
uccuguacugagcugccccgag

SEQ ID NO: 117 - RNA, 21 nt, Homo sapiens
cggggcagcucaguacaggau

SEQ ID NO: 118 - RNA, 88 nt, Homo sapiens
ucucaggcugugacccucuagagggaagcgcuuucuguuggcuaaaagaaaagaaagcgcuucccuucagaguguuaacgcuuugaga

SEQ ID NO: 119 - RNA, 22 nt, Homo sapiens
cucuagagggaagcgcuuucug

SEQ ID NO: 120 - RNA, 21 nt, Homo sapiens
aaagcgcuucccuucagagug

SEQ ID NO: 121 - RNA, 97 nt, Homo sapiens
cugcuccuucucccauacccauugcauaucggaguugugaauucucaaaacaccuccugugugcauggauuacaggagggugagccuugucaucgug

SEQ ID NO: 122 - RNA, 22 nt, Homo sapiens
uacccauugcauaucggaguug

SEQ ID NO: 123 - RNA, 21 nt, Homo sapiens
accuccugugugcauggauua

(19) United States
(12) Patent Application Publication
(10) Pub. No.: US 2015/0232939 A1
(43) Pub. Date: Aug. 20, 2015

(54) MICRO-RNA BIOMARKERS FOR HAEMOLYSIS AND METHODS OF USING SAME

(71) Applicants: Gabriella Sozzi, Milan (IT); Mattia Boeri, Milan (IT)

(72) Inventors: Gabriella Sozzi, Milan (IT); Mattia Boeri, Milan (IT)

(21) Appl. No.: 14/064,109

(22) Filed: Oct. 25, 2013

Related U.S. Application Data
(60) Provisional application No. 61/719,219, filed on Oct. 26, 2012.

(51) Int. Cl. C12Q 1/68

(52) U.S. Cl. CPC C12Q 1/6886; C12Q 2600/178; C12Q 2600/158; C12Q 2600/16

(57) ABSTRACT

The present disclosure relates to micro-RNAs associated with haemolysis and methods of using them. One disclosed method is a procedure for identifying biological samples in which haemolysis is present by assessing the levels of expression of miRNA in the blood or another biological fluid. Also described is a method for identifying individuals at risk of having a disease or disorder associated with haemolysis.

### RELATED APPLICATIONS

This application claims benefit of priority from U.S. Provisional Patent Application 61/719,219, filed Oct. 26, 2012, which is hereby incorporated by reference as if fully set forth.

### FIELD OF THE DISCLOSURE

This disclosure relates to micro-RNA (miRNA) molecules associated with haemolysis, kits comprising them, and methods of using the molecules and kits.

### BACKGROUND OF THE DISCLOSURE

Haemolysis is the rupture of red blood cells. When haemolysis occurs, haemoglobin, which is normally present in high quantities in red blood cells, is released into the blood.

In vitro, haemolysis can occur as a result of the processes of blood collection, transportation, preservation, storage, and other handling processes. In vivo, haemolysis can be caused by a number of conditions, including immune reactions, infections, medications, toxins, and treatments such as use of a heart-lung bypass machine or haemodialysis.

Haemolysis can lead to a condition known as haemolytic anemia, in which red blood cells are destroyed earlier than normal in the lifetime of red blood cells, and the bone marrow is not able to replenish the destroyed red blood cells. As a result, subjects suffering from haemolytic anemia do not have enough healthy red blood cells to carry oxygen to the tissues. One type of haemolytic anemia is immune haemolytic anemia, which occurs when the immune system develops antibodies to attack the body's own red blood cells. This condition can be a sign of other health problems, including but not limited to blood clots, transfusion of blood from a donor with a different blood type, infections, exposure to toxic chemicals, or genetic defects that lead to abnormal blood cell shapes (e.g., thalassemia or sickle cell disease).

Symptoms of haemolytic anemia include grumpiness, weakness, headaches, problems with concentration, brittle nails, light-headedness, pale skin color, shortness of breath, bluish color of the whites of the eyes, and sore tongue.

Early and sensitive detection of haemolysis is important for early diagnosis of haemolytic anemia. Sensitive detection of haemolysis can serve as an early marker for serious conditions or diseases that cause haemolysis, such as but not limited to immune reactions, infections, medications, toxins, and treatments. Additionally, it is important to make sure that blood supplies used for transfusions do not contain a high number of haemolyzed red blood cells, as the haemoglobin released from the ruptured red blood cells can lead to toxicity.

Visual assessment is currently a method commonly used to detect haemolysis, in which redder color in the plasma is an indication of haemolysis. However, this method is not sensitive and it is unreliable, as it is subject to the observer's vision and perception. Another currently used method for detecting haemolysis is the quantification of free haemoglobin in the serum using spectrophotometry. However, this method suffers from the drawback that components, such as lipids, in the serum may lead to signal interference, which decreases sensitivity.

### SUMMARY OF THE DISCLOSURE

The present disclosure is based in part on the principle of detecting haemolysis with high sensitivity and specificity (where specificity is the accuracy of detection of haemolysis). The present disclosure includes a method for detecting haemolysis in a sample. In one embodiment, the disclosed method detects haemolysis in biological samples, such as fluids. In some cases, the fluid is whole blood, a fraction of blood, plasma, or serum.

In other embodiments, the present disclosure provides a method for detecting a subject at risk for haemolysis.

In a first aspect, the disclosure provides a method for detecting haemolysis with high sensitivity and specificity. The method may comprise measuring the level of expression of at least two microRNAs (miRNAs) listed in Table 2 in a biological sample from a subject, comparing the level of expression of said miRNAs from said sample from said subject to the level of expression of said miRNAs from a control biological sample; and determining when the level of expression of said at least two miRNAs in said biological sample deviates with respect to that of the control biological sample. In some embodiments, measuring the expression of at least two miRNAs comprises production of a corresponding cDNA for each miRNA for measurement. In other embodiments, the measuring may comprise labeling each miRNA with a detectable reporter moiety for measurement. In some cases, the labeling may be direct, covalent attachment of the reporter moiety. In other cases, the labeling may be indirect attachment, such as by use of a labeled molecule that is complementary to all or part of the miRNA.

In a second aspect related to the first aspect, the step of determining comprises measuring the level of expression of at least four miRNAs listed in Table 2, and comparing the level of expression of said miRNAs from said sample from said subject to the level of expression of said miRNAs from a control biological sample. In one embodiment, the four miRNAs are miR-16, miR-451, miR-486-5p, and miR-92a. In another embodiment, the four miRNAs are miR-126, miR-15b, miR-221, and miR-30b.

In a third aspect related to the first aspect, the step of determining comprises measuring the level of expression of at least eight miRNAs listed in Table 2, and comparing the level of expression of said miRNAs from said sample from said subject to the level of expression of said miRNAs from a control biological sample. In one embodiment, the eight miRNAs are miR-16, miR-451, miR-486-5p, miR-92a, miR-126, miR-15b, miR-221, and miR-30b.

In one embodiment, the level of expression of miR-16, miR-451, miR-486-5p, and miR-92a is higher in said biological sample than in said control biological sample. In some cases, a level of expression of miR-16, miR-451, miR-486-5p, and miR-92a that is higher in said biological sample than in said control biological sample is used to indicate the presence of haemolysis.

In a fourth aspect related to the first aspect, the step of determining comprises calculating a plurality of ratios or real quotients determined by performing the ratio between the measured values of the levels of expression of a predetermined number of pairs of the miRNA molecules, comparing each of the ratios or real quotients with a respective control value, determining the real quotients which deviate from the respective ratio value or control real quotient.

In one embodiment, the step of determining comprises the steps of: calculating a plurality of real quotients by determining a ratio between the level of expression of at least one pair of miRNAs from at least two miRNAs listed in Table 2, comparing each of the real quotients with a respective control value, and determining the real quotients which deviate from the respective control quotient value. In a further embodiment, the step of determining comprises determining a ratio between the level of expression of at least one pair of miRNAs from at least four miRNAs listed in Table 2. In another embodiment, the step of determining comprises determining a ratio between the level of expression of at least one pair of miRNAs from at least eight miRNAs listed in Table 2. In yet another embodiment, the step of determining comprises determining a ratio between the level of expression of at least one pair of miRNA from at least sixteen miRNAs listed in Table 2.

In a fifth aspect related to the fourth aspect, the step of calculating the plurality of real quotients comprises determining a predetermined number or a predetermined percentage of quotients from among the levels of expression. In one embodiment, the quotients are selected from at least one of the quotients as listed in Table 4. In another embodiment, the quotients are selected from at least eight of the quotients as listed in Table 4. In yet another embodiment, the quotients are selected from at least sixteen of the quotients as listed in Table 4.

In a sixth aspect related to the first aspect, the subject has lung cancer. The type of lung cancer can be but is not limited to small-cell lung cancer (SCLC), non small-cell lung cancer (NSCLC), pulmonary adenocarcinoma (ADC), bronchio-alveolar carcinoma (BAC), squamous-cell lung carcinoma (SeC) or large-cell carcinoma (LC).

In a seventh aspect related to the first aspect, the biological sample is a biological fluid. In some embodiments, the biological fluid is whole blood, a fraction of blood, plasma or serum.

In another embodiment, the biological sample is from a subject with lung cancer. In yet another embodiment, the control biological sample is a biological sample obtained from a disease-free subject. In another embodiment, the control biological sample is a biological sample obtained from a subject that may be diseased but is obtained at a previous time from diagnosis of the disease, such as when the subject was disease-free. In a further embodiment, the control biological sample is obtained from a different tissue from that being tested.

The value of the level of expression of miRNAs is determined by standard methods well known in the art. For example, the means for determining the value of the level of expression is Quantitative Real-time PCR, Microfluidic cards, Microarrays, RT-PCR, quantitative or semi-quantitative, Northern blot, Solution Hybridization, or Sequencing.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents, and other references mentioned herein are incorporated by reference. The references cited herein are not admitted to be prior art to the claimed disclosure. In the case of conflict, the present specification, including definitions, will control. In addition, the materials, methods, and examples are illustrative only and are not intended to be limiting.

Other features and advantages of the disclosure will be apparent from the following detailed description and claims

### Detailed Description of Modes of Practicing the Disclosure

The present disclosure provides methods comprising determining the level of expression of at least two miRNAs, at least four miRNAs, or at least eight miRNAs from the miRNAs listed in Table 2 in a biological sample from a subject, and comparing the level of expression of said miRNAs from said sample from said subject to the level of expression of said miRNA from a control biological sample.

The present disclosure provides methods comprising determining the level of expression of at least two miRNAs, at least four miRNAs, or at least eight miRNAs from the miRNAs listed in Table 2 in a biological sample from a subject, and comparing the level of expression of said miRNAs from said sample from said subject to the level of expression of said miRNAs from a control biological sample, wherein a change or deviation in the level of expression of said at least two miRNAs in said biological sample from said control biological sample identifies the presence of haemolysis in the biological sample.

The present disclosure provides methods comprising determining the level of expression of at least two miRNAs, at least four miRNAs, or at least eight miRNAs from the miRNAs listed in Table 2 in a biological sample from a subject, and comparing the level of expression of said miRNAs from said sample from said subject to the level of expression of said miRNAs from a control biological sample, wherein a change or deviation in the level of expression of said at least two miRNAs in said biological sample from said control biological sample identifies a subject at risk of haemolysis,

Generally, and without limiting the scope of the disclosure, the miRNAs listed in Table 2 may be viewed as including 5 (five) haemolysis-related (HR) miRNAs with p-value of <0.001) and 15 miRNAs that are haemolysis-unrelated (HU) with p-value of >0.05. One or more ratios between one HR miRNA and one HU miRNA can be determined for used in some embodiments of the disclosure. As non-limiting examples, using one HR vs one HU would mean one ratio; one HR vs three HU would mean three ratios; two HR vs two HU would mean four ratios; and three HR vs one HU would mean three ratios. There are 75 possible ratios between 5 HR and 15 HU miRNAs. In some embodiments, 16 ratios from 4 HR miRNAs and 4HU miRNAs are used to detect or identify haemolysis. In some optional embodiments, the expression of one or more of miR-101, miR-142-3p, mir-19b, and miR-660 are not used in the practice of the disclosure.

Thus the methods of the present disclosure can further comprise calculating a plurality of real quotients by determining a ratio between the level of expression of at least two miRNAs, at least four miRNAs, or at least eight miRNAs listed in Table 2; comparing each of the real quotients with a respective control value; and determining the real quotients which deviate from the respective control quotient value.

The methods of the present disclosure can further comprise determining a number or percentage of real quotients which deviate from the respective control value.

Calculating the plurality of real quotients comprises using the expression level of at least two miRNAs, at least four miRNAs, or at least eight miRNAs from the miRNAs listed in Table 2.

Calculating the plurality of real quotients comprises determining a predetermined number or a predetermined percentage of quotients from among the levels of expression, wherein the quotients are selected from at least one of the quotients, at least two of the quotients, at least four, at least eight, or at least sixteen of the quotients, as listed in Table 4. In one embodiment, at least 2 out of 16 ratios or real quotients shown in Table 4 that deviate from respective control values indicates the presence of haemolysis. In another embodiment, at least 4 out of 16 ratios or real quotients shown in Table 4 that deviate from respective control values indicates the presence of haemolysis. In a further embodiment, at least 8 out of 16 ratios or real quotients shown in Table 4 that deviate from respective control values indicates the presence of haemolysis.

The methods of the present disclosure can further comprise detecting haemolysis in a subject that has lung cancer.

The lung cancer can be small-cell lung cancer (SCLC), non small-cell lung cancer (NSCLC), pulmonary adenocarcinoma (ADC), bronchio-alveolar carcinoma (BAC), squamous-cell lung carcinoma (SCC) or large-cell carcinoma (LCLC).

The biological sample may be a biological fluid. The biological fluid can be whole blood, a fraction of blood, plasma or serum. The biological sample originates from a smoker individual who, at the moment of the collection of the sample, does not present lung cancer if subjected to imaging diagnostic methods, in particular the smoker individual not presenting nodules of dimensions of greater than 5 mm if subjected to a spiral computed tomagraph (CT) scan.

The control biological sample is a biological sample from a disease-free subject. The control biological sample is a biological sample obtained from said subject at a previous time. The control biological sample is obtained from said subject up to three years preceding diagnosis or when the subject was disease-free. The control biological sample is a biological sample obtained from a different tissue from said subject.

Preferably, the subject is a mammal. The mammal can be e.g., any mammal, e.g., a human, primate, mouse, rat, dog, cat, cow, horse, goat, camel, sheep or a pig. Preferably, the mammal is a human. In other embodiments, the subject is a bird or fowl.

As used herein, microRNA or miRNA is small, non-coding, RNA molecules (length 19-25 nucleotides). In particular, reference is made to miRNA present in biological samples of human tissue, for example whole blood, plasma, serum, saliva, bronchial secretion, or bronchial condensate or breath condensate.

Table 1 provides a summary of the miRNAs for use in the present disclosure.

TABLE 1

| miRNA Name | Sequence |
|---|---|
| hsa-miR-7-2 | CUGGAUACAGAGUGGACCGGCUGGCCCCAUCUGGAAGACUAGUGA |
| (pre-miR) | UUUUGUUGUUGUCUUACUGCGCUCAACAACAAAUCCCAGUCUACC |
| | UAAUGGUGCCAGCCAUCGCA (SEQ ID NO: 1) |
| hsa-miR-7-2-5p | UGGAAGACUAGUGAUUUUGUUGU (SEQ ID NO: 2) |
| (mature miR 5′ arm) | |
| hsa-miR-7-2-3p | CAACAAAUCCCAGUCUACCUAA (SEQ ID NO: 3) |
| (mature miR 3′ arm) | |
| hsa-miR-15b | UUGAGGCCUUAAAGUACUGUAGCAGCACAUCAUGGUUUACAUGCU |
| (pre-miR) | ACAGUCAAGAUGCGAAUCAUUAUUUGCUGCUCUAGAAAUUUAAGG |
| | AAAUUCAU (SEQ ID NO: 4) |
| hsa-miR-15b-5p | UAGCAGCACAUCAUGGUUUACA (SEQ ID NO: 5) |
| (mature miR 5′ arm) | |
| hsa-miR-15b-3p | CGAAUCAUUAUUUGCUGCUCUA (SEQ ID NO: 6) |
| (mature miR 3′ arm) | |
| hsa-miR-16-1 | GUCAGCAGUGCCUUAGCAGCACGUAAAUAUUGGCGUUAAGAUUCU |
| (pre-miR from Chr. 13) | AAAAUUAUCUCCAGUAUUAACUGUGCUGCUGAAGUAAGGUUGAC |
| | (SEQ ID NO: 7) |
| hsa-miR-16-2 | GUUCCACUCUAGCAGCACGUAAAUAUUGGCGUAGUGAAAUAUAUA |
| (pre-miR from Chr. 3) | UUAAACACCAAUAUUACUGUGCUGCUUUAGUGUGAC (SEQ ID |
| | NO: 8) |
| hsa-miR-16-5p | UAGCAGCACGUAAAUAUUGGCG (SEQ ID NO: 9) |
| (mature miR 5′ arm) | |
| hsa-miR-16-3p | CCAAUAUUACUGUGCUGCUUUA (SEQ ID NO: 10) |
| (mature miR 3′ arm) | |
| hsa-miR-17 | GUCAGAAUAAUGUCAAAGUGCUUACAGUGCAGGUAGUGAUAUGUG |
| (pre-miR) | CAUCUACUGCAGUGAAGGCACUUGUAGCAUUAUGGUGAC (SEQ |
| | ID NO: 11) |
| hsa-miR-17-5p | CAAAGUGCUUACAGUGCAGGUAG (SEQ ID NO: 12) |
| (mature miR 5′ arm) | |
| hsa-miR-17-3p | ACUGCAGUGAAGGCACUUGUAG (SEQ ID NO: 13) |
| (mature miR 3′ arm) | |
| hsa-miR-19b-1 | CACUGUUCUAUGGUUAGUUUUGCAGGUUUGCAUCCAGCUGUGUGA |
| (pre-miR from Chr. 13) | UAUUCUGCUGUGCAAAUCCAUGCAAAACUGACUGUGGUAGUG |
| | (SEQ ID NO: 14) |
| hsa-miR-19b-1-5p | AGUUUUGCAGGUUUGCAUCCAGC (SEQ ID NO: 15) |
| (mature miR 5′ arm | |
| from Chr. 13) | |
| hsa-miR-19b-2 | ACAUUGCUACUUACAAUUAGUUUUGCAGGUUUGCAUUUCAGCGUA |
| (pre-miR from Chr. X) | UAUAUGUAUAUGUGGCUGUGCAAAUCCAUGCAAAACUGAUUGUGA |
| | UAAUGU (SEQ ID NO: 16) |
| hsa-miR-19b-2-5p | AGUUUUGCAGGUUUGCAUUUCA (SEQ ID NO: 17) |
| (mature miR 5′ arm | |
| from Chr. X) | |
| hsa-miR-19b-3p | UGUGCAAAUCCAUGCAAAACUGA (SEQ ID NO: 18) |
| (mature miR 3′ arm | |
| from Chr. 13 or X) | |
| hsa-miR-21 | UGUCGGGUAGCUUAUCAGACUGAUGUUGACUGUUGAAUCUCAUGG |
| (pre-miR) | CAACACCAGUCGAUGGGCUGUCUGACA (SEQ ID NO: 19) |
| hsa-miR-21-5p | UAGCUUAUCAGACUGAUGUUGA (SEQ ID NO: 20) |
| (mature miR 5′ arm) | |
| hsa-miR-21-3p | CAACACCAGUCGAUGGGCUGU (SEQ ID NO: 21) |
| (mature miR 3′ arm) | |
| hsa-miR-28 | GGUCCUUGCCCUCAAGGAGCUCACAGUCUAUUGAGUUACCUUUCU |
| (pre-miR) | GACUUUCCCACUAGAUUGUGAGCUCCUGGAGGGCAGGCACU |
| | (SEQ ID NO: 22) |
| hsa-miR-28-5p | AAGGAGCUCACAGUCUAUUGAG (SEQ ID NO: 23) |
| (mature miR 5′ arm) | |
| hsa-miR-28-3p | CACUAGAUUGUGAGCUCCUGGA (SEQ ID NO: 24) |
| (mature miR 3′ arm) | |
| hsa-miR-30a | GCGACUGUAAACAUCCUCGACUGGAAGCUGUGAAGCCACAGAUGG |
| (pre-miR) | GCUUUCAGUCGGAUGUUUGCAGCUGC (SEQ ID NO: 25) |
| hsa-miR-30a-5p | UGUAAACAUCCUCGACUGGAAG (SEQ ID NO: 26) |
| (mature miR 5′ arm) | |
| hsa-miR-30a-3p | CUUUCAGUCGGAUGUUUGCAGC (SEQ ID NO: 27) |
| (mature miR 3′ arm) | |
| hsa-miR-30b | ACCAAGUUUCAGUUCAUGUAAACAUCCUACACUCAGCUGUAAUAC |
| (pre-miR) | AUGGAUUGGCUGGGAGGUGGAUGUUUACUUCAGCUGACUUGGA |
| | (SEQ ID NO: 28) |
| hsa-miR-30b-5p | UGUAAACAUCCUACACUCAGCU (SEQ ID NO: 29) |
| (mature miR 5′ arm) | |
| hsa-miR-30b-3p | CUGGGAGGUGGAUGUUUACUUC (SEQ ID NO: 30) |
| (mature miR 3′ arm) | |
| hsa-miR-30c-1 | ACCAUGCUGUAGUGUGUGUAAACAUCCUACACUCUCAGCUGUGAG |
| (pre-miR from Chr. 1) | CUCAAGGUGGCUGGGAGAGGGUUGUUUACUCCUUCUGCCAUGGA |
| | (SEQ ID NO: 31) |
| hsa-miR-30c-1-3p | CUGGGAGAGGGUUGUUUACUCC (SEQ ID NO: 32) |
| (mature miR 3′ arm | |
| from Chr. 1) | |
| hsa-miR-30c-2 | AGAUACUGUAAACAUCCUACACUCUCAGOUGUGGAAAGUAAGAAA |
| (pre-miR from Chr. 6) | GCUGGGAGAAGGCUGUUUACUCUUUCU (SEQ ID NO: 33) |
| hsa-miR-30c-5p | UGUAAACAUCCUACACUCUCAGC (SEQ ID NO: 34) |
| (mature miR 5′ arm | |
| from Chr. 1 or 6) | |
| hsa-miR-30c-2-3p | CUGGGAGAAGGCUGUUUACUCU (SEQ ID NO: 35) |
| (mature miR 3′ arm | |
| from Chr. 6) | |
| hsa-miR-30d | GUUGUUGUAAACAUCCCCGACUGGAAGCUGUAAGACACAGCUAAG |
| (pre-miR) | CUUUCAGUCAGAUGUUUGCUGCUAC (SEQ ID NO: 36) |
| hsa-miR-30d-5p | UGUAAACAUCCCCGACUGGAAG (SEQ ID NO: 37) |
| (mature miR 5′ arm) | |
| hsa-miR-30d-3p | CUUUCAGUCAGAUGUUUGCUGC (SEQ ID NO: 38) |
| (mature miR 3′ arm) | |
| hsa-miR-34b | GUGCUCGGUUUGUAGGCAGUGUCAUUAGCUGAUUGUACUGUGGUG |
| (pre-miR) | GUUACAAUCACUAACUCCACUGCCAUCAAAACAAGGCAC (SEQ |
| | ID NO: 39) |
| hsa-miR-34b-5p | UAGGCAGUGUCAUUAGCUGAUUG (SEQ ID NO: 40) |
| (mature miR 5′ arm) | |
| hsa-miR-34b-3p | CAAUCACUAACUCCACUGCCAU (SEQ ID NO: 41) |
| (mature miR 3′ arm) | |
| hsa-mirR-92a-1 | CUUUCUACACAGGUUGGGAUCGGUUGCAAUGCUGUGUUUCUGUAU |
| (pre-miR from Chr. 13) | GGUAUUGCACUUGUCCCGGCCUGUUGAGUUUGG (SEQ ID |
| | NO: 42) |
| hsa-miR-92a-1-5p | AGGUUGGGAUCGGUUGCAAUGCU (SEQ ID NO: 43) |
| (mature miR 5′ arm | |
| from Chr. 13) | |
| hsa-miR-92a-3p | UAUUGCACUUGUCCCGGCCUGU (SEQ ID NO: 44) |
| (mature miR 3′ arm | |
| from Chr. 13 or X) | |
| hsa-miR-92a-2 | UCAUCCCUGGGUGGGGAUUUGUUGCAUUACUUGUGUUCUAUAUAA |
| (pre-miR from Chr. X) | AGUAUUGCACUUGUCCCGGCCUGUGGAAGA (SEQ ID NO: 45) |
| hsa-miR-92a-2-5p | GGGUGGGGAUUUGUUGCAUUAC (SEQ ID NO: 46) |
| (mature miR 5′ arm | |
| from Chr. X) | |
| hsa-miR-101-1 | UGCCCUGGCUCAGUUAUCACAGUGCUGAUGCUGUCUAUUCUAAAG |
| (pre-miR from Chr. 1) | GUACAGUACUGUGAUAACUGAAGGAUGGCA (SEQ ID NO: 47) |
| hsa-miR-101-5p | CAGUUAUCACAGUGCUGAUGCU (SEQ ID NO: 48) |
| (mature miR 5′ arm | |
| from Chr. 1 or 9) | |
| hsa-miR-101-1-3p | UACAGUACUGUGAUAACUGAA (SEQ ID NO: 49) |
| (mature miR 3′ arm | |
| from Chr. 1) | |
| hsa-miR-101-2 | ACUGUCCUUUUUCGGUUAUCAUGGUACCGAUGCUGUAUAUCUGAA |
| (pre-miR from Chr. 9) | AGGUACAGUACUGUGAUAACUGAAGAAUGGUGGU (SEQ ID |
| | NO: 50) |
| hsa-miR-101-2-3p | UACAGUACUGUGAUAACUGAA (SEQ ID NO: 51) |
| (mature miR 3′ arm | |
| from Chr. 9) | |
| hsa-miR-106a | CCUUGGCCAUGUAAAAGUGCUUACAGUGCAGGUAGCUUUUUGAGA |
| (pre-miR) | UCUACUGCAAUGUAAGCACUUCUUACAUUACCAUGG (SEQ ID |
| | NO: 52) |
| hsa-miR-106a-5p | AAAAGUGCUUACAGUGCAGGUAG (SEQ ID NO: 53) |
| (mature miR 5′ arm) | |
| hsa-miR 106a-3p | CUGCAAUGUAAGCACUUCUUAC (SEQ ID NO: 54) |
| (mature miR 3′ arm) | |
| hsa-miR-126 | CGCUGGCGACGGGACAUUAUUACUUUUGGUACGCGCUGUGACACU |
| (pre-miR) | UCAAACUCGUACCGUGAGUAAUAAUGCGCCGUCCACGGCA (SEQ |
| | ID NO: 55) |
| hsa-miR-126-5p | CAUUAUUACUUUUGGUACGCG (SEQ ID NO: 56) |
| (mature miR 5′ arm) | |
| hsa-miR-126-3p | UCGUACCGUGAGUAAUAAUGCG (SEQ ID NO: 57) |
| (mature miR 3′ arm) | |
| hsa-miR-133a-1 | ACAAUGCUUUGCUAGAGCUGGUAAAAUGGAACCAAAUCGCCUCUU |
| (pre-miR from Chr. 18) | CAAUGGAUUUGGUCCCCUUCAACCAGCUGUAGCUAUGCAUUGA |
| | (SEQ ID NO: 58) |
| hsa-miR I33a-2 | GGGAGCCAAAUGCUUUGCUAGAGCUGGUAAAAUGGAACCAAAUCG |
| (pre-miR from Chr. 20) | ACUGUCCAAUGGAUUUGGUCCCCUUCAACCAGCUGUAGCUGUGCA |
| | UUGAUGGCGCCG (SEQ ID NO: 59 ) |
| hsa-miR-133a | UUUGGUCCCCUUCAACCAGCUG (SEQ ID NO: 60) |
| (mature miR 3′ arm | |
| from Chr. 18 or 20) | |
| hsa-miR-140 | UGUGUCUCUCUCUGUGUCCUGCCAGUGGUUUUACCCUAUGGUAGG |
| (pre-miR) | UUACGUCAUGCUGUUCUACCACAGGGUAGAACCACGGACAGGAUA |
| | CCGGGGCACC (SEQ ID NO: 61) |
| hsa-miR-140-5p | CAGUGGUUUUACCCUAUGGUAG (SEQ ID NO: 62) |
| (mature miR 5′ arm) | |
| hsa-miR-140-3p | UACCACAGGGUAGAACCACGG (SEQ ID NO: 63) |
| (mature miR 3′ arm) | |
| hsa-miR-142 | GACAGUGCAGUCACCCAUAAAGUAGAAAGCACUACUAACAGCACU |
| (pre-miR) | GGAGGGUGUAGUGUUUCCUACUUUAUGGAUGAGUGUACUGUG |
| | (SEQ ID NO: 64) |
| hsa-miR-142-5p | CAUAAAGUAGAAAGCACUACU (SEQ ID NO: 65) |
| (mature miR 5′ arm) | |
| hsa-miR-142-3p | UGUAGUGUUUCCUACUUUAUGGA (SEQ ID NO: 66) |
| (mature miR 3′ arm) | |
| hsa-miR- 144 | UGGGGCCCUGGCUGGGAUAUCAUCAUAUACUGUAAGUUUGCGAUG |
| (pre-miR) | AGACACUACAGUAUAGAUGAUGUACUAGUCCGGGCACCCCC |
| | (SEQ ID NO: 67) |
| hsa-miR-144-5p | GGAUAUCAUCAUAUACUGUAAG (SEQ ID NO: 68) |
| (mature miR 5′ arm) | |
| hsa-miR-144-3p | UACAGUAUAGAUGAUGUACU (SEQ ID NO: 69) |
| (mature miR 3′ arm) | |
| hsa-miR-145 | CACCUUGUCCUCACGGUCCAGUUUUCCCAGGAAUCCCUUAGAUGC |
| (pre-miR) | UAAGAUGGGGAUUCCUGGAAAUACUGUUCUUGAGGUCAUGGUU |
| | (SEQ ID NO: 70) |
| hsa-miR-145-5p | GUCCAGUUUUCCCAGGAAUCCCU (SEQ ID NO: 71) |
| (mature miR 5′ arm) | |
| hsa-miR-145-3p | GGAUUCCUGGAAAUACUGUUCU (SEQ ID NO: 72) |
| (mature miR 3′ arm) | |
| hsa-miR-148a | GAGGCAAAGUUCUGAGACACUCCGACUCUGAGUAUGAUAGAAGUC |
| (pre-miR) | AGUGCACUACAGAACUUUGUCUC (SEQ ID NO: 73) |
| hsa-miR-148a-5p | AAAGUUCUGAGACACUCCGACU (SEQ ID NO: 74) |
| (mature miR 5′ arm) | |
| hsa-miR-148a-3p | UCAGUGCACUACAGAACUUUGU (SEQ ID NO: 75) |
| (mature miR 3′ arm) | |
| hsa-miR-197 | GGCUGUGCCGGGUAGAGAGGGCAGUGGGAGGUAAGAGCUCUUCAC |
| (pre-miR) | CCUUCACCACCUUCUCCACCCAGCAUGGCC (SEQ ID NO: 76) |
| hsa-miR-197-5p | CGGGUAGAGAGGGCAGUGGGAGG (SEQ ID NO: 77) |
| (mature miR 5′ arm) | |
| hsa-miR-197-3p | UUCACCACCUUCUCCACCCAGC (SEQ ID NO: 78) |
| (mature miR 3′ arm) | |
| hsa-miR-200b | CCAGCUCGGGCAGCCGUGGCCAUCUUACUGGGCAGCAUUGGAUGG |
| (pre miR) | AGUCAGGUCUCUAAUACUGCCUGGUAAUGAUGACGGCGGAGCCCU |
| | GCACG (SEQ ID NO: 79) |
| hsa-miR-200b-5p | CAUCUUACUGGGCAGCAUUGGA (SEQ ID NO: 80) |
| (mature miR 5′ arm) | |
| hsa-miR-200b-3p | UAAUACUGCCUGGUAAUGAUGA (SEQ ID NO: 81) |
| (mature miR 3′ arm) | |
| hsa-miR-205 | AAAGAUCCUCAGACAAUCCAUGUGCUUCUCUUGUCCUUCAUUCCA |
| (pre-miR) | CCGGAGUCUGUCUCAUACCCAACCAGAUUUCAGUGGAGUGAAGUU |
| | CAGGAGGCAUGGAGCUGACA (SEQ ID NO: 82) |
| hsa-miR-205-5p | UCCUUCAUUCCACCGGAGUCUG (SEQ ID NO: 83) |
| (mature miR 5′ arm) | |
| hsa-miR-205-3p | GAUUUCAGUGGAGUGAAGUUC (SEQ ID NO: 84) |
| (mature miR 3′ arm) | |
| hsa-miR-210 | ACCCGGCAGUGCCUCCAGGCGCAGGGCAGCCCCUGCCCACCGCAC |
| (pre-miR) | ACUGCGCUGCCCCAGACCCACUGUGCGUGUGACAGCGGCUGAUCU |
| | GUGCCUGGGCAGCGCGACCC (SEQ ID NO: 85) |
| hsa-miR-210 | CUGUGCGUGUGACAGCGGCUGA (SEQ ID NO: 86) |
| (mature miR) | |
| hsa-miR-219-1 | CCGCCCCGGGCCGCGGCUCCUGAUUGUCCAAACGCAAUUCUCGAG |
| (pre-miR) | UCUAUGGCUCCGGCCGAGAGUUGAGUCUGGACGUCCCGAGCCGCC |
| | GCCCCCAAACCUCGAGCGGG (SEQ ID NO: 87) |
| hsa-miR-219-1-5p | UGAUUGUCCAAACGCAAUUCU (SEQ ID NO: 88) |
| (mature miR 5′ arm) | |
| hsa-miR-219-1-3p | AGAGUUGAGUCUGGACGUCCCG (SEQ ID NO: 89) |
| (mature miR 3′ arm) | |
| hsa-miR-221 | UGAACAUCCAGGUCUGGGGCAUGAACCUGGCAUACAAUGUAGAUU |
| (pre-miR) | UCUGUGUUCGUUAGGCAACAGCUACAUUGUCUGCUGGGUUUCAGG |
| | CUACCUGGAAACAUGUUCUC (SEQ ID NO: 90) |
| hsa-miR-221-5p | ACCUGGCAUACAAUGUAGAUUU (SEQ ID NO: 91) |
| (mature miR 5′ arm) | |
| hsa-miR-221-3p | AGCUACAUUGUCUGCUGGGUUUC (SEQ ID NO: 92) |
| (mature miR 3′ arm) | |
| hsa-miR-320a | GCUUCGCUCCCCUCCGCCUUCUCUUCCCGGUUCUUCCCGGAGUCG |
| (pre-miR) | GGAAAAGCUGGGUUGAGAGGGCGAAAAAGGAUGAGGU (SEQ ID |
| | NO: 93) |
| hsa-miR-320a | AAAAGCUGGGUUGAGAGGGCGA (SEQ ID NO: 94) |
| (mature miR) | |
| hsa-miR-320b-1 | AAUUAAUCCCUCUCUUUCUAGUUCUUCCUAGAGUGAGGAAAAGCU |
| (pre-miR from Chr. 1:117214371- | GGGUUGAGAGGGCAAACAAAUUAACUAAUUAAUU (SEQ ID |
| 117214449) | NO: 95) |
| hsa-miR-320b-2 | UGUUAUUUUUUGUCUUCUACCUAAGAAUUCUGUCUCUUAGGCUUU |
| (pre-miR from Chr. 1:224444706- | CUCUUCCCAGAUUUCCCAAAGUUGGGAAAAGCUGGGUUGAGAGGG |
| 224444843) | CAAAAGGAAAAAAAAAGAAUUCUGUCUCUGACAUAAUUAGAUAGG |
| | GAA (SEQ ID NO: 96) |
| hsa-miR-320b | AAAAGCUGGGUUGAGAGGGCAA (SEQ ID NO: 97) |
| (mature miR from Chr. 1) | |
| hsa-miR-320c-1 | UUUGCAUUAAAAAUGAGGCCUUCUCUUCCCAGUUCUUCCCAGAGU |
| (pre-miR from Chr. 18:19263471- | CAGGAAAAGCUGGGUUGAGAGGGUAGAAAAAAAAUGAUGUAGG |
| 19263558) | (SEQ ID NO: 98) |
| hsa-miR-320c-2 | CUUCUCUUUCCAGUUCUUCCCAGAAUUGGGAAAAGCUGGGUUGAG |
| (pre-miR from Chr. 18-21901650- | AGGGU (SEQ ID NO: 99) |
| 21901699) | |
| hsa-miR-320-c | AAAAGCUGGGUUGAGAGGGU (SEQ ID NO: 100) |
| (mature miR from either Chr 18 | |
| loci) | |
| hsa-miR-320d-1 | UUCUCGUCCCAGUUCUUCCCAAAGUUGAGAAAAGCUGGGUUGAGA |
| (pre-miR from Chr. 13) | GGA (SEQ ID NO: 101) |
| hsa-miR-320d-2 | UUCUCUUCCCAGUUCUUCUUGGAGUCAGGAAAAGCUGGGUUGAGA |
| (pre-miR from Chr. X) | GGA (SEQ ID NO: 102) |
| hsa-miR-320d | AAAAGCUGGGUUGAGAGGA (SEQ ID NO: 103) |
| (mature miR from Chr. 13 or X) | |
| hsa-miR-320e | GCCUUCUCUUCCCAGUUCUUCCUGGAGUCGGGGAAAAGCUGGGUU |
| (pre-miR) | GAGAAGGU (SEQ ID NO: 104) |
| hsa-miR-320e | AAAGCUGGGUUGAGAAGG (SEQ ID NO: 105) |
| (mature miR) | |
| hsa-miR-324 | CUGACUAUGCCUCCCCGCAUCCCCUAGGGCAUUGGUGUAAAGCUG |
| (pre-miR) | GAGACCCACUGCCCCAGGUGCUGCUGGGGGUUGUAGUC (SEQ |
| | ID NO: 106) |
| hsa-miR-324 | CGCAUCCCCUAGGGCAUUGGUGU (SEQ ID NO: 107) |
| (mature miR 5′ arm) | |
| hsa-miR-324 | ACUGCCCCAGGUGCUGCUGG (SEQ ID NO: 108) |
| (mature miR 3′ arm) | |
| hsa-miR-429 | CGCCGGCCGAUGGGCGUCUUACCAGACAUGGUUAGACCUGGCCCU |
| (pre-miR) | CUGUCUAAUACUGUCUGGUAAAACCGUCCAUCCGCUGC (SEQ |
| | ID NO: 109) |
| hsa-miR-429 | UAAUACUGUCUGGUAAAACCGU (SEQ ID NO: 110) |
| (mature at) | |
| hsa-miR-451a | CUUGGGAAUGGCAAGGAAACCGUUACCAUUACUGAGUUUAGUAAU |
| (pre-miR) | GGUAAUGGUUCUCUUGCUAUACCCAGA (SEQ ID NO: 111) |
| hsa-miR-451a | AAACCGUUACCAUUACUGAGUU (SEQ ID NO: 112) |
| (mature miR) | |
| hsa-miR-451b | UGGGUAUAGCAAGAGAACCAUUACCAUUACUAAACUCAGUAAUGG |
| (pre-miR) | UAACGGUUUCCUUGCCAUUCCCA (SEQ ID NO: 113) |
| hsa-miR-451b | UAGCAAGAGAACCAUUACCAUU (SEQ ID NO: 114) |
| (mature miR) | |
| hsa-miR-486 | GCAUCCUGUACUGAGCUGCCCCGAGGCCCUUCAUGCUGCCCAGCU |
| (pre-miRNA) | CGGGGCAGCUCAGUACAGGAUAC (SEQ ID NO: 115) |
| hsa-miR-486-5p | UCCUGUACUGAGCUGCCCCGAG (SEQ ID NO: 116) |
| (mature miR 5′ arm) | |
| hsa-miR-486-3p | CGGGGCAGCUCAGUACAGGAU (SEQ ID NO: 117) |
| (mature miR 3′ arm) | |
| hsa-miR-518e | UCUCAGGCUGUGACCCUCUAGAGGGAAGCGCUUUCUGUUGGCUAA |
| (pre-miR) | AAGAAAAGAAAGCGCUUCCCUUCAGAGUGUUAACGCUUUGAGA |
| | (SEQ ID NO: 118) |
| hsa-miR-518e Sp | CUCUAGAGGGAAGCGCUUUCUG (SEQ ID NO: 119) |
| (mature miR 5′ arm) | |
| hsa-miR-518e-3p | AAAGCGCUUCCCUUCAGAGUG (SEQ ID NO: 120) |
| (mature miR 3′ arm) | |
| hsa-miR-660 | CUGCUCCUUCUCCCAUACCCAUUGCAUAUCGGAGUUGUGAAUUCU |
| (pre-miR) | CAAAACACCUCCUGUGUGCAUGGAUUACAGGAGGGUGAGCCUUGU |
| | CAUCGUG (SEQ ID NO: 121) |
| hsa-miR-660-5p | UACCCAUUGCAUAUCGGAGUUG (SEQ ID NO: 122) |
| (mature miR 5′ arm) | |
| hsa-miR-660-3p | ACCUCCUGUGUGCAUGGAUUA (SEQ ID NO: 123) |
| (mature miR 3′ arm) | |

The present disclosure also provides, in part, a kit for performing the disclosed methods. The kit may include a specific binding agent that selectively binds to a disclosed miRNA, and instructions for carrying out a method as described herein.

As used herein the term “sample” refers to anything which may contain a disclosed miRNA. In many cases, the miRNA is a cell-free nucleic acid molecule. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18^{th} edition (1990). These texts can, of course, also be referred to in making or using an aspect of the disclosure.

Other features and advantages of the present disclosure are apparent from the different examples. The provided examples illustrate different components and methodology useful in practicing the present disclosure. The examples do not limit the disclosure.

### EXAMPLES

### Example 1

### Detection of Haemolysis Using a microRNA (miRNA) Signature

### Plasma Collection

Samples of whole blood were collected, with addition of EDTA, and stored at room temperature for no longer than 1-2 hours before processing. Storage at reduced temperature is to be avoided because it may lead to non-specific release of miRNA. The samples were centrifuged at approximately 1250 times g at 4° C. for 10 minutes to separate plasma, which was carefully transferred while avoiding material closest to the lymphocytic ring. The plasma was centrifuged again under the same conditions and then separated into aliquots, with avoidance of pelleted material, that were stored at −80° C. for up to 1 year or longer.

miRNA Expression Levels in Haemolysed Versus Non-Haemolysed Samples

To develop a miRNA signature for detection of haemolysis, 24 plasma samples were haemolysed. Haemolysis was assessed by visual inspection. Haemolysed samples were profiled for expression of a panel of miRNAs using custom made microfluidic cards. Expression of the miRNAs in the haemolysed samples was compared to that in non-haemolysed (based on visual assessment) samples from 98 disease-free individuals.

Four miRNAs (miR-16, miR-451, miR-486-5p, and miR-92a) were included in the development of the miRNA signature. Raw Ct values (a measure of expression level) are shown in Table 2. It is apparent that the 4 miRNAs, miR-16, miR-451, miR-486-5p and miR-92a, in addition to miR-140-3p, are significantly upregulated (p<0.001) in haemolysed samples versus non-haemolysed samples.

TABLE 2
Raw Ct values for miRNAs in haemolysed versus
non-haemolysed samples

| | NOT | | | | | |
|---|---|---|---|---|---|---|
| | HAEMOLYSED | | HAEMOLYSED | | | |
| | Ct | | Ct | | | |
| | average | s.d. | average | s.d. | p-value | Δ |
| miR-101 | 30.0 | 1.5 | 28.8 | 1.8 | 0.007 | 1.2 |
| miR-106a | 20.5 | 1.4 | 19.9 | 1.6 | 0.092 | 0.6 |
| miR-126 | 21.3 | 1.4 | 21.7 | 1.5 | 0.261 | −0.4 |
| miR-133a | 30.0 | 1.8 | 30.8 | 2.4 | 0.132 | −0.8 |
| miR-140-3p | 30.7 | 1.8 | 28.9 | 1.9 | <0.001 | 1.8 |
| miR-140-5p | 25.8 | 1.3 | 25.8 | 1.6 | 0.862 | −0.1 |
| miR-142-3p | 21.5 | 1.5 | 22.3 | 1.8 | 0.030 | −0.9 |
| miR-145 | 26.1 | 1.6 | 26.6 | 1.8 | 0.260 | −0.5 |
| miR-148a | 28.9 | 1.5 | 28.6 | 1.5 | 0.486 | 0.2 |
| miR-15b | 24.9 | 1.5 | 25.0 | 1.6 | 0.823 | −0.1 |
| miR-16 | 20.7 | 1.5 | 18.3 | 2.2 | <0.001 | 2.4 |
| miR-17 | 20.6 | 1.4 | 20.0 | 1.6 | 0.078 | 0.7 |
| miR-197 | 26.1 | 1.5 | 26.2 | 1.2 | 0.659 | −0.1 |
| miR-19b | 21.5 | 1.5 | 20.4 | 1.8 | 0.006 | 1.2 |
| miR-21 | 24.8 | 1.4 | 24.9 | 1.9 | 0.886 | −0.1 |
| miR-221 | 24.1 | 1.4 | 24.8 | 1.9 | 0.108 | −0.7 |
| miR-28-3p | 26.4 | 1.5 | 26.8 | 1.4 | 0.256 | −0.4 |
| miR-30b | 22.2 | 1.5 | 22.8 | 1.6 | 0.087 | −0.6 |
| miR-30c | 24.0 | 1.6 | 24.3 | 1.6 | 0.294 | −0.4 |
| miR-320 | 23.1 | 1.4 | 22.6 | 1.4 | 0.107 | 0.5 |
| miR-451 | 22.9 | 1.6 | 20.0 | 2.5 | <0.001 | 2.9 |
| miR-486-5p | 23.5 | 1.6 | 20.6 | 2.2 | <0.001 | 2.8 |
| miR-660 | 29.2 | 1.5 | 27.8 | 2.1 | 0.004 | 1.5 |
| miR-92a | 24.0 | 1.4 | 22.8 | 1.5 | <0.001 | 1.3 |

Generation of miRNA Ratios as Signatures for Haemolysis

In order to discriminate between haemolysed versus non-haemolysed samples using miRNA ratios, normal plasma samples containing serial dilutions of an in vitro haemolysed plasma sample were analyzed for miRNA expression levels. Ratios of various miRNAs to each of the four miRNAs shown in Table 2 to be upregulated (miR-16, miR-451, miR-486-5p, and miR-92a) were calculated. MiR-126, miR-15b, miR-221, and miR-30b, were identified as the miRNAs whose ratios with the four upregulated miRNAs had the best correlation with haemolysis (Table 3).

TABLE 3
Plasma miRNA expression level ratios representing haemolysis in vitro¹

| | Cut- | | | | | | | | | Not |
|---|---|---|---|---|---|---|---|---|---|---|
| | off | 1/800 | 1/1600 | 1/3200 | 1/6400 | 1/12800 | 1/25600 | 1/51200 | 1/102400 | treated |
| 451/126 | 0.74 | 2.60 | 1.66 | 0.70 | 0.94 | 0.41 | 0.28 | −0.53 | −0.69 | −0.78 |
| 451/15b | 4.07 | 5.88 | 5.28 | 4.30 | 4.54 | 4.00 | 4.05 | 3.11 | 3.39 | 2.85 |
| 451/221 | 3.81 | 5.69 | 4.94 | 4.14 | 4.16 | 3.76 | 3.66 | 3.32 | 3.06 | 2.26 |
| 451/30b | 1.53 | 2.73 | 2.15 | 1.25 | 1.12 | 0.85 | 0.50 | −0.31 | −0.01 | −0.29 |
| 486- | −0.34 | 2.25 | 1.47 | 0.41 | 0.33 | −0.11 | −0.02 | −0.27 | −0.75 | −1.00 |
| 5p/126 | | | | | | | | | | |
| 486- | 3.33 | 5.51 | 5.09 | 4.01 | 3.93 | 3.48 | 3.75 | 3.38 | 3.33 | 2.63 |
| 5p/15b | | | | | | | | | | |
| 486- | 2.63 | 5.32 | 4.74 | 3.85 | 3.55 | 3.23 | 3.36 | 3.59 | 2.99 | 2.04 |
| 5p/221 | | | | | | | | | | |
| 486- | 0.70 | 2.36 | 1.96 | 0.96 | 0.52 | 0.33 | 0.20 | −0.05 | −0.07 | −0.51 |
| 5p/30b | | | | | | | | | | |
| 92a/126 | −1.86 | −0.54 | −1.09 | −1.84 | −1.84 | −1.80 | −1.93 | −2.47 | −2.77 | −2.70 |
| 92a/15b | 1.70 | 2.74 | 2.53 | 1.76 | 1.76 | 1.79 | 1.83 | 1.18 | 1.31 | 0.93 |
| 92a/221 | 1.08 | 2.55 | 2.18 | 1.60 | 1.38 | 1.55 | 1.45 | 1.39 | 0.97 | 0.34 |
| 92a/30b | −0.83 | −0.41 | −0.60 | −1.29 | −1.66 | −1.36 | −1.71 | −2.24 | −2.10 | −2.22 |
| 16/126 | 1.92 | 5.15 | 4.05 | 3.54 | 3.50 | 3.00 | 3.26 | 2.91 | 2.42 | 1.79 |
| 16/15b | 5.34 | 8.42 | 7.67 | 7.14 | 7.10 | 6.59 | 7.03 | 6.55 | 6.50 | 5.43 |
| 16/221 | 5.01 | 8.28 | 7.32 | 6.98 | 6.72 | 6.35 | 6.65 | 6.76 | 6.16 | 4.84 |
| 16/30b | 2.75 | 5.28 | 4.54 | 4.09 | 3.68 | 3.44 | 3.49 | 3.13 | 3.10 | 2.28 |
| Ratios | | 16 | 16 | 13 | 13 | 10 | 9 | 8 | 6 | 1 |
| exceeding | | | | | | | | | | |
| cut-off | | | | | | | | | | |
| R414/375 | | 2.48 | 2.02 | 1.80 | 1.61 | 1.49 | 1.44 | 1.45 | 1.39 | 1.34 |

¹Values are expressed as log₂(ratio)

Mean baseline miRNA ratios were calculated using the formula, mean ratio+1.5 s.d. (standard deviation), from non-haemolysed plasma samples from 98 disease-free individuals. These baseline ratios were set as the cut-off for each of the 16 miRNA ratios in developing the miRNA signature for haemolysis.

Using the miRNA Signature to Detect Haemolysis

The miRNA signature can be used to detect haemolysis in plasma samples. Table 4 shows a list of 16 ratios composed of 8 miRNAs (miR-126, 15b, 30b, 221, 451, 16, 486-5p and 92a). In particular, the 4 miRNAs, miR-451, miR-486-5p, miR-16 and miR-92a, are all overexpressed in haemolysed samples. This overexpression can be used as an indicator of haemolysis in a plasma sample. In addition, if 8, or more, out of 16 ratios shown in Table 4 exceed the cut-off value, then the plasma sample is positive for haemolysis

TABLE 4
miRNA signature for haemolysis

| Ratios of | Cut-off |
|---|---|
| miRNAs | (log2) |
| 126/451 | <−0.74 |
| 15b/451 | <−4.07 |
| 221/451 | <−3.81 |
| 30b/451 | <−1.53 |
| 126/486 | <0.34 |
| 15b/486 | <−3.33 |
| 221/486 | <−2.63 |
| 30b/486 | <−0.70 |
| 126/92a | <1.86 |
| 15b/92a | <−1.70 |
| 221/92a | <−1.08 |
| 30b/92a | <0.83 |
| 126/16 | <−1.92 |
| 15b/16 | <−5.34 |
| 221/16 | <−5.01 |
| 30b/16 | <−2.75 |

The ratios in Table 3 are inverted (and so the cut-offs have a sign change but the same numerical value) in Table 4. The same inversion with a sign change may be made in the practice of the disclosed methods. All disclosed cut-off values are non-limiting and may change somewhat with further analysis. Additionally, the disclosed methods may be performed with the use of some or all of the representative ratios, as well as other available ratios of miRNAs in Table 2, as described herein. In some embodiments of the disclosure, variations of the disclosed cut-offs are used. Non-limiting examples include a variation of the numerical value of any cut-off by +/−0.5% or thereabouts, +/−1.0% or thereabouts, +/−2.0% or thereabouts, +/−3.0% or thereabouts, +/−4.0% or thereabouts, +/−5.0% or thereabouts, +/−6.0% or thereabouts, +/−7.0% or thereabouts, +/−8.0% or thereabouts, +/−9.0% or thereabouts, or +/−10.0% or thereabouts.

### Example 2

### Comparison of miRNA Signature Versus Haemoglobin Absorbance Methods for Detecting Haemolysis

Haemoglobin is known in the art to have an absorbance wavelength at 414 nm One standard method used in both scientific and clinical practice to analyze haemolysis is the spectroscopic measurement at the wavelength of 414 nm (using an absorbance threshold of 0.2) of free haemoglobin in plasma samples. In this experiment, the absorbance at 414 nm was normalized against the absorbance at 375 nm in order to overcome the high background signal in some samples (e.g., in lipemic samples). A cut-off of 1.4 for the ratio of absorbance at 414 nm to absorbance at 375 nm was set.

As shown in the last row of Table 3, samples with a value of A414 nm/A375 nm greater than 1.40 corresponded to a miRNA signature wherein at least 8 out of 16 miRNA ratios exceeded their respective cut-offs. These settings were used to evaluate the power of the miRNA signature in distinguishing the 24 haemolysed from 98 non-haemolysed (based on visual inspection) plasma samples. The miRNA signature method exhibited a sensitivity of 0.88 and a specificity of 0.93. In regard to sensitivity, it was clear that the 24 samples that were visually assessed to be haemolysed were indeed haemolysed. However, in regard to specificity, it was unclear based solely on visual inspection whether some of the 98 non-haemolysed samples in fact contained some haemolysis that was not detectable visually.

For this reason, the two methods (miRNA signature and spectrophotometry) were compared using an independent series composed of a selection of 60 plasma samples that were visually haemolysed (red, orange or dark yellow) and 43 plasma samples non-haemolysed based on visual inspection. Concerning sensitivity (Table 5), the 5 most haemolysed (red) samples were all recognized as positive for haemolysis by both methods. However, in evaluating the orange and dark yellow samples, the miRNA signature method was more sensitive (>90% versus 76% for the spectrophotometric method).

TABLE 5
Comparing spectrophotometer and miRNAs results to evaluate
haemolysis in 60 visually haemolysed plasma samples¹

| | Red | Orange | Dark yellow |
|---|---|---|---|
| Total | 5 | 25 | 30 |
| R414/375 | 5 (100.0) | 19 (76.0) | 23 (76.7) |
| miRNAs | 5 (100.0) | 23 (92.0) | 29 (96.7) |
| Concordance | 5 (100.0) | 21 (84.0) | 22 (73.3) |

¹( ) percentages of the total that were detected by each method as being haemolysed

On the other hand, in the 43 plasma samples that were non-haemolysed based on visual inspection (Table 6), results showed that 3 (7%) samples were positive for haemolysis using both methods, 1 (2%) sample was positive in only the spectrophotometric method, and 2 (5%) samples were positive in only the miRNA signature method. Forty (93%) samples had an agreement in haemolysis classification between the two methods. Excluding the 3 cases positive in both the methods, the spectrophotometric method had 97.5% specificity and the miRNA signature method had 95% specificity.

TABLE 6
Comparing spectrophotometer and miRNAs results
to evaluate haemolysis in 43 non-haemolysed (based
on visual inspection) plasma samples¹

| | miRNAs | | |
|---|---|---|---|
| R414/375 | Positive | Negative | Tot |
| Positive | 3 (7.0) | 1 (2.3) | 4 (9.3) |
| Negative | 2 (4.7) | 37 (86.0) | 39 (90.7) |
| Tot | 5 (11.6) | 38 (88.4) | 40 (93.0) |

¹( ) percentages of the total; Kappa statistic = 0.63

The citation of documents herein is not to be construed as reflecting an admission that any is relevant prior art. Moreover, their citation is not an indication of a search for relevant disclosures. All statements regarding the date(s) or contents of the documents is based on available information and is not an admission as to their accuracy or correctness.

All references cited herein, including patents, patent applications, and publications, are hereby incorporated by reference in their entireties, whether previously specifically incorporated or not.

Having now fully described the inventive subject matter, it will be appreciated by those skilled in the art that the same can be performed within a wide range of equivalent parameters, concentrations, and conditions without departing from the spirit and scope of the disclosure and without undue experimentation.

While this disclosure has been described in connection with specific embodiments thereof, it will be understood that it is capable of further modifications. This application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

We claim:

1. A method comprising
a) determining the level of expression of at least two miRNAs listed in Table 2 in a biological sample from a subject, and
b) comparing the level of expression of said miRNAs from said sample from said subject to the level of expression of said miRNAs from a control biological sample,
wherein a change or deviation in the level of expression of said at least two miRNAs in said biological sample from said control biological sample determines the presence of haemolysis.

2. The method of claim 1, comprising determining the level of expression of at least four miRNAs listed in Table 2.

3. The method of claim 2, wherein the at least four miRNAs are
miR-16, miR-451, miR-486-5p, and miR-92a; or
miR-126, miR-15b, miR-221, and miR-30b.

4. The method of claim 1, comprising determining the level of expression of at least eight miRNAs listed in Table 2.

5. The method of claim 4, wherein the at least eight miRNAs are miR-16, miR-451, miR-486-5p, miR-92a, miR-126, miR-15b, miR-221, and miR-30b.

6. The method of claim 3, wherein the level of expression of miR-16, miR-451, miR-486-5p, and miR-92a is higher in said biological sample than in said control biological sample.

7. The method of claim 1, further comprising
calculating a plurality of real quotients by determining a ratio between the level of expression of at least one pair of miRNAs from at least two miRNAs listed in Table 2;
comparing each of the real quotients with a respective control value; and
determining the real quotients which deviate from the respective control quotient value.

8. The method of claim 7, comprising determining a ratio between the level of expression of
at least one pair of miRNAs from at least four miRNAs listed in Table 2; or
at least one pair of miRNAs from at least eight miRNAs listed in Table 2; or
at least one pair of miRNAs from at least sixteen miRNAs listed in Table 2.

9. The method of claim 7, wherein calculating the plurality of real quotients comprises determining a predetermined number or a predetermined percentage of quotients from among the levels of expression, wherein the quotients are selected from at least one of the quotients as listed in Table 4.

10. The method of claim 9, wherein the quotients are selected from
at least eight of the quotients as listed in Table 4; or
at least sixteen of the quotients as listed in Table 4.

11. The method of claim 1, wherein the subject has lung cancer.

12. The method of claim 11, wherein the lung cancer is small-cell lung cancer (SCLC), non small-cell lung cancer (NSCLC), pulmonary adenocarcinoma (ADC), bronchio-alveolar carcinoma (BAC), squamous-cell lung carcinoma (SCC) or large-cell carcinoma (LCLC).

13. The method of claim 1, wherein the biological sample is a biological fluid.

14. The method of claim 13, wherein the biological fluid is whole blood, a fraction of blood, plasma or serum.

15. The method of claim 1, wherein said control biological sample is a biological sample from a disease-free subject.

16. The method of claim 1, wherein said control biological sample is a biological sample obtained from said subject at a previous time.

17. The method of claim 1, wherein said control biological sample is a biological sample obtained from a different tissue from said subject.

18. The method of claim 1, wherein the means for determining the value of the level of expression is selected from the group consisting of Quantitative Real-time PCR, Microfluidic cards, Microarrays, RT-PCR, quantitative or semi-quantitative, Northern blot, Solution Hybridization, and Sequencing.

19. The method of claim 1, wherein the determining comprises production of a cDNA molecule complementary to at least a portion of each said at least two miRNAs.

20. The method of claim 19, wherein the cDNA molecules comprise heterologous sequences, optionally adapter sequence(s), linked to sequence from said at least two miRNAs.
